(19) United States
(12) Patent Application Publication
(10) Pub. No.: US 2020/0360405 A1
(43) Pub. Date: Nov. 19, 2020

(54) COMPOSITIONS AND METHODS FOR REDUCING LOCAL AND SYSTEMIC RISKS OF ENVENOMATION

(71) Applicant: THE HENRY M. JACKSON FONDATION FOR THE ADVANCEMENT OF MILITARY MEDICINE, INC., Bethesda, MD (US)

(72) Inventor: Angela JOCKHECK-CLARK, San Antonio, TX (US)

(73) Assignee: The Henry M. Jackson Foundation for the Advancement of Midicine, Inc., Bethesda, MD (US)

(21) Appl. No.: 16/606,347

(22) PCT Filed: Apr. 17, 2018
(86) PCT No.: PCT/US2018/027977

§ 371 Date: Oct. 18, 2019

Related U.S. Application Data
(60) Provisional application No. 62/487,390, filed on Apr. 19, 2017.

(51) Int. Cl. A61K 31/65; A61K 31/28; A61K 31/198; A61P 39/02; A61K 39/395

(52) U.S. Cl. CPC A61K 31/65; A61K 31/28; A61K 39/39533; A61P 39/02; A61K 39/39575; A61K 31/198

(57) ABSTRACT

Methods of treating at least one condition associated with envenomation, pharmaceutical compositions, and kits comprising a first compound having formula (I): and a second compound chosen from N-acetyl-L-cysteine, sodium aurothiomalate, silibinin, sodium copper chlorophyllin, and pharmaceutically acceptable salts and solvates thereof.

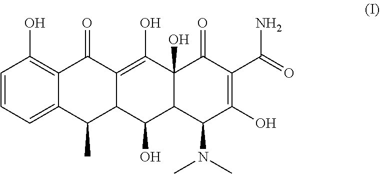 [SMILES: C[C@H]1c2cccc(O)c2C(=O)C2=C(O)[C@]3(O)C(=O)C(C(N)=O)=C(O)[C@@H](N(C)C)C3[C@@H](O)C21]

This application claims the benefit of priority of U.S. provisional application No. 62/487,390, filed Apr. 19, 2017, which is incorporated herein by reference in its entirety.

The invention was made with government support under Grant W91 1QY-1 1-D-0053 awarded by the United States Army Medical Research and Materiel Command. The government has certain rights in the invention.

Disclosed herein are novel methods for treating at least one condition associated with envenomation, comprising administering a first compound and a second compound. Also disclosed herein are novel compositions and kits.

Envenomation can cause a variety of severe local and systemic effects that can necessitate extensive medical attention. Snake venoms, for example, are complex compositions of proteins, enzymes, lipids, amines, and target-specific toxins that vary significantly by species. Unlike target-specific toxins that are unique to the venom of a particular species, many venoms contain similar types of spreading factors. These spreading factors may include enzymatic compounds that cause general tissue disruption and permit the spreading of other venom components. In some embodiments, a particular venom may contain one or more spreading factors from one or more classes. For example, one or more spreading factors from one or more classes may be found in the venom of various species of a snake; bee; stonefish; scorpion; spider; lizard; wasp; hornet; sea urchin; cnidarian such as, e.g., an anemone, coral, common or tropical jellyfish; or a toxic plant, such as, e.g., poison ivy, poison sumac, or poison oak.

Spreading factors are generally divided into three classes of compounds commonly found in individual venoms: hyaluronidases, metalloproteinases, and phospholipase A2 (PLA2). Hyaluronidase is a non-toxic endoglycosidase that degrades hyaluronan and the connective tissues surrounding blood vessels. Metalloproteinases, such as those in some snake venoms, may hydrolyze endothelial barriers surrounding blood vessels, resulting in hemorrhagic activities. Lastly, PLA2 belongs to a diverse family of lipolytic enzymes with a variety of structural and functional specificities, and are known to hydrolyze membrane lipids and induce necrotic cell death. PLA2 is prevalent in the venom of, e.g., snakes of the Elapidae and Viperidae families and some jellyfish, spiders, and scorpions. Venoms from various species can therefore induce both rapid dissemination of venom components in a subject and cause immediate and continuing damage to affected tissues (e.g., necrosis and/or nerve damage).

Within minutes of being bitten by, e.g., a snake, spreading factors in the venom may begin to distort the extracellular matrix and may cause local effects such as, e.g., edema, blistering, hemorrhaging, tissue necrosis, and/or nerve damage. Ultimately, these spreading factors may degrade the endothelium around blood vessels, allowing life-threatening, target-specific venom components to spread systemically, which may result in myotoxicity, cardiotoxicity, and/or alterations in hematological systems.

Once the venom accesses the blood stream, traditional anti-venom treatments may be used to neutralize target-specific venom proteins. However, antibody-based antivenoms cannot diffuse into the tissues that have suffered damage, which may result in persistent and potentially permanent tissue damage, e.g., necrosis and/or nerve damage, requiring extensive medical attention. Species-specific antibody-based antivenoms are also frequently required, which can necessitate the stocking of many different antivenoms in hospital and other treatment settings.

Thus, there exists a need to develop universal methods and compositions for preventing or reducing the spread and diffusion of venom from the site of envenomation and reducing the damage caused by spreading factors at or near the site of envenomation. Furthermore, there exists a need for an envenomation treatment that is applicable to a variety of envenomation sources. For instance, there is a need for treatments effective against envenomation from a snake; bee; stonefish; scorpion; spider; lizard; wasp; hornet; sea urchin; cnidarian such as, e.g., an anemone, coral, common or a tropical jellyfish; or a toxic plant, such as, e.g., poison ivy, poison sumac, or poison oak. Such treatment would be beneficial because some venoms can result in life-threatening conditions once in the bloodstream and/or cause persistent local tissue disruption and/or necrosis. Such a treatment may enhance the post-envenomation survival rate by, e.g., inhibiting spreading factors and/or otherwise slowing the spread of venom. Similarly, such a treatment may ameliorate continuing or latent symptoms such as, e.g., ongoing necrosis of an envenomed tissue. This may be particularly beneficial in situations where treatment is required without knowledge of the identity or source of envenomation.

Disclosed in various embodiments herein are combination therapies using a first compound having formula (I) or a pharmaceutically acceptable salt or solvate thereof, and a second compound comprising N-acetyl-L-cysteine, sodium aurothiomalate, silibinin, and/or sodium copper chlorophyllin, or a pharmaceutically acceptable salt or solvate thereof. it was surprisingly discovered that in some embodiments, the combinations are effective to treat, reduce, ameliorate, or counteract at least one condition associated with envenomation. The combinations may be effective against envenomation from more than one species.

In various embodiments, the first compound is

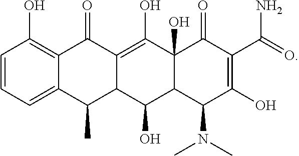 [SMILES: C[C@H]1c2cccc(O)c2C(=O)C2=C(O)[C@]3(O)C(=O)C(C(N)=O)=C(O)[C@@H](N(C)C)C3[C@@H](O)C21]

In some embodiments, the first compound is

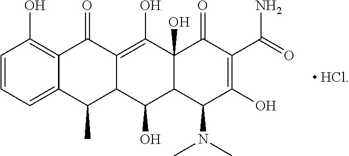 [SMILES: C[C@H]1c2cccc(O)c2C(=O)C2=C(O)[C@]3(O)C(=O)C(C(N)=O)=C(O)[C@@H](N(C)C)C3[C@@H](O)C21]

In some embodiments, the first compound is

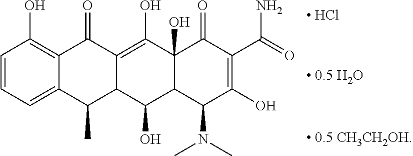

In some embodiments, the first compound is a tetracycline derivative. In certain embodiments, the tetracycline derivative is selected from tetracycline, minocycline, doxycycline, demeclocycline, and tigecycline. In various embodiments, the second compound is a salt or solvate of a compound listed above.

In some embodiments, the second compound is N-acetyl-L-cysteine. In some embodiments, the second compound is sodium aurothiomalate. In some embodiments, the second compound is silibinin. In other embodiments, the second compound is sodium copper chlorophyllin. In some embodiments, the second compound itself comprises a combination of two or more of N-acetyl-L-cysteine, sodium aurothiomalate, silibinin, and/or sodium copper chlorophyllin. In some embodiments, the second compound comprises a combination of N-acetyl-L-cysteine and sodium aurothiomalate. In various embodiments, the second compound is a salt or solvate of a compound listed above.

In some embodiments, disclosed herein is a method of treating at least one condition associated with envenomation in a subject comprising administering to a subject in need thereof a therapeutically effective amount of a first compound having formula (I):

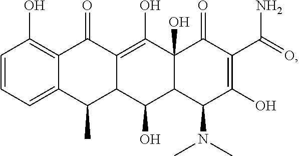 [SMILES: C[C@H]1c2cccc(O)c2C(=O)C2=C(O)[C@]3(O)C(=O)C(C(N)=O)=C(O)[C@@H](N(C)C)C3[C@@H](O)C21]

or a pharmaceutically acceptable salt or solvate thereof; a therapeutically effective amount of a second compound chosen from N-acetyl-L-cysteine, sodium aurothiomalate, silibinin, sodium copper chlorophyllin, and pharmaceutically acceptable salts and solvates thereof; and at least one pharmaceutically acceptable vehicle.

In some embodiments, disclosed herein is a pharmaceutical composition comprising: a first compound having formula (I):

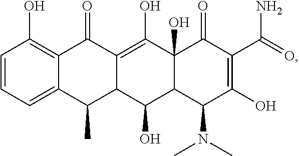 [SMILES: C[C@H]1c2cccc(O)c2C(=O)C2=C(O)[C@]3(O)C(=O)C(C(N)=O)=C(O)[C@@H](N(C)C)C3[C@@H](O)C21]

or a pharmaceutically acceptable salt or solvate thereof; a second compound chosen from N-acetyl-L-cysteine and sodium aurothiomalate or a pharmaceutically acceptable salt or solvate thereof; and at least one pharmaceutically acceptable vehicle.

In some embodiments, disclosed herein is a kit comprising: a first compound having formula (I):

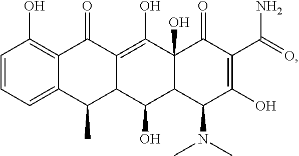 [SMILES: C[C@H]1c2cccc(O)c2C(=O)C2=C(O)[C@]3(O)C(=O)C(C(N)=O)=C(O)[C@@H](N(C)C)C3[C@@H](O)C21]

or a pharmaceutically acceptable salt or solvate thereof; and a second compound chosen from N-acetyl-L-cysteine and sodium aurothiomalate or a pharmaceutically acceptable salt or solvate thereof.

Other aspects and embodiments of the present disclosure are set forth or will be readily apparent from the following description. It is to be understood that both the foregoing general description and the following detailed description are exemplary and explanatory only, and are not intended to be restrictive of the claims.

### BRIEF DESCRIPTION OF THE DRAWINGS

FIG. 1 shows the inhibition of Naja naja kaouthia and Vipera russeiii crude venom hyaluronidase activities.

FIG. 2 shows the inhibition of N. kaouthia and V. russeiii crude venom gelatinase activities.

FIG. 3 shows the inhibition of N. kaouthia and V. russeiii crude venom PLA2 activities.

FIG. 4 shows the effects of a combination of doxycycline and sodium aurothiomalate on N. kaouthia and V. russeiii spreading factor activities, including those that were elevated in the presence of the individual drugs.

FIG. 5 shows the effects of a combination of doxycycline and N-acetyl-L-cysteine on N. kaouthia and V. russelli spreading factor activities.

FIG. 6 shows the inhibitory activity of sodium aurothiomalate and manoalide toward V. russelli hyaluronidase activities and V. russelli and N. kaouthia PLA2 activities.

### DETAILED DESCRIPTION

Disclosed herein, in various embodiments, are combinations comprising a first compound and a second compound to treat envenomation. In some embodiments, the combination is administered to a subject to treat, ameliorate, reduce, and/or prevent at least one condition associated with envenomation. In some embodiments, a first and second compound are provided for use in treating or preventing at least one condition associated with envenomation. In some embodiments, a first and second compound are used in the manufacture of a medicament for treating or preventing at least one condition associated with envenomation.

In some embodiments, the envenomation may be from a bee sting (e.g., a honey bee), a wasp sting, a hornet sting, a scorpion sting, a spider bite (e.g., spiders of the genus Loxosceles), a jellyfish sting (e.g., the jellyfish Nemopilema nomuai), and/or a snake bite. In some embodiments, the envenomation may be from a cnidarian family members or a pathogenic bacteria, such as, e.g., staphylococcus, streptococci, anthrax, Clostridium, or E. coli. In some embodiments, the envenomation from the cnidarian family members or the pathogenic bacteria may be a pore-forming toxin, also known as a porin.

In some embodiments, the envenomation may be from a bite of a spider. In some embodiments, the bite is from a Widow spider, a Recluse spider, or a Hobo spider.

In some embodiments, envenomation may be from a bite of a snake. In some embodiments, the snake is from the family Elapidae, Viperidae, Colubridae, Hydrophiidae, or Atractaspididae. In some embodiments, the snake is from the Colubridae, Hydrophiidae, or Atractaspididae family. In some embodiments, the snake bite is from a cobra. In some embodiments, the snake bite is from a snake of the Elapidae or Viperidae family. In some embodiments, the snake is a Copperhead snake, a Pygmy Rattlesnake, a Cottonmouth snake, an Eastern Diamondback Rattlesnake, a Timber Rattlesnake, or an Eastern Coral snake.

In some embodiments, the at least one condition associated with envenomation is one or more of edema, blistering, hemorrhage, tissue necrosis, damage to nerve terminals, myotoxicity, cardiotoxicity, pulmonary dysfunction (including pulmonary edema), and/or alterations in one or more hematological systems or pathways. In some embodiments, the at least one condition associated with envenomation is identified by an increase in one or more myonecrosis and/or serum myonecrosis markers, and/or a change in one or more hemorrhagic indicators.

In some embodiments, the at least one condition associated with envenomation comprises edema. In some embodiments, the at least one condition associated with envenomation comprises blistering. In some embodiments, the at least one condition associated with envenomation comprises hemorrhage. In some embodiments, the at least one condition associated with envenomation comprises tissue necrosis. In some embodiments, the at least one condition associated with envenomation comprises damage to nerve terminals. In some embodiments, the at least one condition associated with envenomation comprises myotoxicity. In some embodiments, the at least one condition associated with envenomation comprises cardiotoxicity. In some embodiments, the at least one condition associated with envenomation comprises alterations in hematological systems. In some embodiments, the at least one condition associated with envenomation comprises an increase in one or more myonecrosis and/or serum myonecrosis markers, and/or a change in one or more hemorrhagic indicators. In some embodiments, the at least one condition associated with envenomation comprises an increase in hemorrhagic indicators. In some embodiments, the at least one condition associated with envenomation comprises a combination of two or more conditions mentioned above.

### Compounds and Administration

In various embodiments, the first compound is

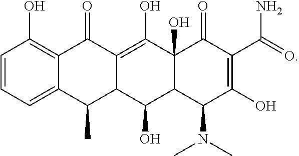 [SMILES: C[C@H]1c2cccc(O)c2C(=O)C2=C(O)[C@]3(O)C(=O)C(C(N)=O)=C(O)[C@@H](N(C)C)C3[C@@H](O)C21]

In some embodiments, the first compound is

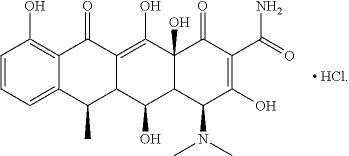 [SMILES: C[C@H]1c2cccc(O)c2C(=O)C2=C(O)[C@]3(O)C(=O)C(C(N)=O)=C(O)[C@@H](N(C)C)C3[C@@H](O)C21]

In some embodiments, the first compound is

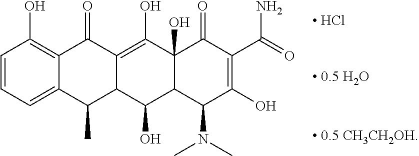 [SMILES: C[C@H]1c2cccc(O)c2C(=O)C2=C(O)[C@]3(O)C(=O)C(C(N)=O)=C(O)[C@@H](N(C)C)C3[C@@H](O)C21 ; "· HCl"]

In some embodiments, the first compound is a tetracycline derivative. In certain embodiments, the tetracycline derivative is selected from a tetracycline derivative, a minocycline, a doxycycline, a demeclocycline, and a tigecycline. In various embodiments, the first compound is a salt or solvate of a compound listed above.

In some embodiments, the second compound is N-acetyl-L-cysteine. In some embodiments, the second compound is sodium aurothiomalate. In some embodiments, the second compound is silibinin. In other embodiments, the second compound is sodium copper chlorophyllin. In some embodiments, the second compound itself comprises a combination of two or more of N-acetyl-L-cysteine, sodium aurothiomalate, silibinin, and/or sodium copper chlorophyllin. In some embodiments, the second compound comprises a combination of N-acetyl-L-cysteine and sodium aurothiomalate. In various embodiments, the second compound is a salt or solvate of a compound listed above.

In some embodiments, the first compound and the second compound may be administered within about 96, 84, 72, 60, 48, 36, 24, 12, 6, 5, 4, 3, or 2 hours, or 60, 50, 40, 30, 20, 10, 5, or fewer minutes of envenomation (or any time period in between). In some embodiments, the first compound and the second compound may be administered at any time point during the spreading of venom following an envenomation event. In some embodiments, the first compound and the second compound may be administered within about one hour of envenomation. In other embodiments, the first compound and the second compound are administered within about thirty minutes of envenomation. In one embodiment, the first compound and the second compound are administered within about fifteen minutes of envenomation. In one embodiment, the first compound and the second compound are administered within about 12 hours of envenomation. In one embodiment, the first compound and the second compound are administered within about 24 hours of envenomation. In one embodiment, the first compound and the second compound are administered within about 96 hours of envenomation.

In some embodiments, the first compound and the second compound are administered at a time point after envenomation that significantly reduces the spread and/or diffusion of venom from the site of envenomation, and/or significantly reduces damage (e.g., necrotic damage) to tissue exposed to the venom, as compared to the spread and/or damage in the absence of treatment or after an antibody-based anti-venom treatment. For instance, the first compound and the second compound may be administered within 24 hours (e.g., 24, 18, 12, 6, 3, 2, 1, or fewer hours) of envenomation to reduce the spread and/or distribution of venom from the site of envenomation. As another example, treatment of envenomation may comprise administering the first compound and the second compound before and/or after administering an antibody-based anti-venom treatment to reduce or minimize necrotic damage of tissue exposed to the venom.

In some embodiments, the first compound is administered before, at the same time as, or after administration of the second compound. In one embodiment, the first compound is administered before the second compound. In some embodiments, the first compound is administered about thirty minutes before the second compound is administered. In some embodiments, the first compound is administered about 24 hours, 12 hours, 6 hours, 3 hours, 2 hours, or 60, 50, 40, 30, 20, 10, 5, or fewer minutes before the second compound (or any time period in between). In some embodiments, the first compound is administered at approximately the same time as the second compound. In some embodiments, the first compound and the second compound are administered simultaneously. In one embodiment, the first compound is administered in a bolus, vial, or mixture also containing the second compound. In some embodiments, the first compound is administered after the second compound is administered. In one embodiment, the first compound is administered about thirty minutes after the second compound is administered. In some embodiments, the first compound is administered about 24 hours, 12 hours, 6 hours, 3 hours, 2 hours, or 60, 50, 40, 30, 20, 10, 5, or fewer minutes after the second compound (or any time period in between).

In some embodiments, the first compound and second compound are administered before, at the same time as, or after the administration of another therapeutic agent. In some embodiments, the other therapeutic agent is an anti-inflammatory agent, a steroid, a coagulant, an antimicrobial agent, or an antivenom agent (e.g., an antibody-based antivenom).

In some embodiments, the first compound and second compound are administered before, at the same time as, or after the administration of an antibody-based antivenom. In one embodiment, the first compound and the second compound are administered before the administration of the antibody-based antivenom. In some embodiments, the first compound and the second compound are administered about 48, 36, 24, 18, 6, 3, 2, or 1 hours, or 60, 50, 40, 30, 20, 10, 5, or fewer minutes before administering the antibody-based antivenom (or any time period in between). In some embodiments, the first compound and the second compound are administered at the same time as the antibody-based anti-venom. In one embodiment, the first compound and the second compound are administered after the administration of the antibody-based antivenom. In some embodiments, the first compound and the second compound are administered about 48, 36, 24, 18, 6, 3, 2, or 1 hours, or 60, 50, 40, 30, 20, 10, 5, or fewer minutes after administering the antibody-based antivenom (or any time period in between).

In some embodiments, the first compound and second compound are administered before, at the same time as, or after the administration of an antimicrobial agent. In one embodiment, the first compound and the second compound are administered before the administration of the antimicrobial agent. In some embodiments, the first compound and the second compound are administered 48, 36, 24, 18, 6, 3, 2, or 1 hours, or 60, 50, 40, 30, 20, 10, 5, or fewer minutes before administering the antimicrobial agent (or any time period in between). In some embodiments, the first compound and the second compound are administered at the same time as the antimicrobial agent. In one embodiment, the first compound and the second compound are administered after administering the antimicrobial agent. In some embodiments, the first compound and the second compound are administered 48, 36, 24, 18, 6, 3, 2, or 1 hours, or 60, 50, 40, 30, 20, 10, 5, or fewer minutes after administering the antimicrobial agent (or any time period in between).

Timing, sequence, and frequency of administration of the compositions disclosed herein may be implemented by one of ordinary skill in the art. A non-limiting list of factors that may influence the timing, sequence, and frequency of administration of the compositions disclosed herein includes venom source, identity of the first compound, identity of the second compound, health of the envenomed subject, whether the composition will be administered in conjunction with any other therapeutic agents, and the like.

### Pharmaceutical Compositions and Preparations

In various embodiments, a pharmaceutical composition is provided, comprising a first compound having formula (I):

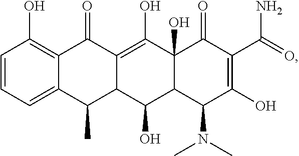 [SMILES: C[C@H]1c2cccc(O)c2C(=O)C2=C(O)[C@]3(O)C(=O)C(C(N)=O)=C(O)[C@@H](N(C)C)C3[C@@H](O)C21]

or a pharmaceutically acceptable salt or solvate thereof; a second compound chosen from N-acetyl-L-cysteine, sodium aurothiomalate, silibinin, sodium copper chlorophyllin, and a pharmaceutically acceptable salt or solvate thereof; and at least one pharmaceutically acceptable vehicle. In some embodiments, the second compound comprises N-acetyl-L-cysteine and/or sodium aurothiomalate. In some embodiments, the pharmaceutical composition further comprises a pharmaceutically acceptable carrier. In some embodiments, the pharmaceutical composition further comprises a pharmaceutically acceptable excipient.

In various embodiments, a kit composition is provided, comprising a first compound having formula (I):

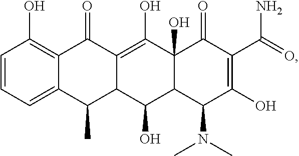 [SMILES: C[C@H]1c2cccc(O)c2C(=O)C2=C(O)[C@]3(O)C(=O)C(C(N)=O)=C(O)[C@@H](N(C)C)C3[C@@H](O)C21]

or a pharmaceutically acceptable salt or solvate thereof; a second compound chosen from N-acetyl-L-cysteine, sodium aurothiomalate, silibinin, sodium copper chlorophyllin, and a pharmaceutically acceptable salt or solvate thereof; and at least one pharmaceutically acceptable vehicle. In some embodiments, the second compound comprises N-acetyl-L-cysteine and/or sodium aurothiomalate.

In some embodiments, the first compound may be administered in an amount ranging from about 0.01 g to about 25 g. In some embodiments, the first compound may be administered in an amount ranging from about 0.01 g to about 20 g. In some embodiments, the first compound may be administered in an amount ranging from about 0.1 g to about 10 g. In some embodiments, the first compound may be administered in an amount ranging from about 0.1 g to about 1 g. In some embodiments, the first compound may be administered in an amount ranging from about 0.5 g to about 1.5 g. In some embodiments, the first compound is administered in an amount of about 0.1 g, about 0.2 g, about 0.3 g, about 0.4 g, about 0.5 g, about 0.75 g, about 1 g, about 1.25 g, about 1.5 g, or any amount in between.

In some embodiments, when the second compound is N-acetyl-L-cysteine, the second compound is administered in an amount ranging from about 0.5 g to about 300 g. In some embodiments, when the second compound is N-acetyl-L-cysteine, the second compound is administered in an amount ranging from about 0.5 g to about 200 g. In some embodiments, when the second compound is N-acetyl-L-cysteine, the second compound is administered in an amount ranging from about 5 g to about 150 g. In some embodiments, when the second compound is N-acetyl-L-cysteine, the second compound is administered in an amount ranging from about 5 g to about 100 g. In some embodiments, when the second compound is N-acetyl-L-cysteine, the second compound is administered in an amount ranging from about 7 g to about 27 g. In some embodiments, when the second compound is N-acetyl-L-cysteine, the second compound is administered in an amount ranging from about 18 g to about 54 g. In some embodiments, when the second compound is N-acetyl-L-cysteine, the second compound is administered in an amount of about 7 g, about 10 g, about 15 g, about 20 g, about 25 g, about 18 g, about 23 g, about 28 g, about 33 g, about 38 g, about 43 g, about 48 g, about 54 g, or any amount in between.

In some embodiments, when the second compound is sodium aurothiomalate, the second compound is administered in an amount ranging from about 0.05 g to about 25 g. In some embodiments, when the second compound is sodium aurothiomalate, the second compound is administered in an amount ranging from about 0.05 g to about 15 g. In some embodiments, when the second compound is sodium aurothiomalate, the second compound is administered in an amount ranging from about 0.05 g to about 10 g. In some embodiments, when the second compound is sodium aurothiomalate, the second compound is administered in an amount ranging from about 0.1 g to about 5 g. In some embodiments, when the second compound is sodium aurothiomalate, the second compound is administered in an amount ranging from about 0.1 g to about 1 g. In some embodiments, when the second compound is sodium aurothiomalate, the second compound is administered in an amount ranging from about 0.1 g to about 0.5 g. In some embodiments, when the second compound is sodium aurothiomalate, the second compound is administered in an amount ranging from about 0.25 g to about 0.75 g. In some embodiments, when the second compound is sodium aurothiomalate, the second compound is administered in an amount of about 0.05 g, about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, or any amount in between.

Effective doses of the first compound and the second compound may be calculated according to a variety of factors, such as, for example, route of administration, age, condition, body weight and/or infected body surface area of the envenomed person. Similarly, effective doses of the first compound and the second compound may also be calculated according to the location of the envenomed tissue, the envenomation source, and/or the amount of venom to which the envenomed person was exposed.

### Benefits of Combination Therapy

In various embodiments, administering a combination of a first compound having formula (I):

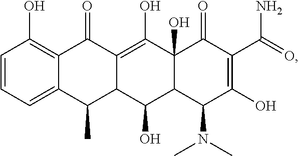 [SMILES: C[C@H]1c2cccc(O)c2C(=O)C2=C(O)[C@]3(O)C(=O)C(C(N)=O)=C(O)[C@@H](N(C)C)C3[C@@H](O)C21]

or a pharmaceutically acceptable salt or solvate thereof; and a second compound chosen from N-acetyl-L-cysteine, sodium aurothiomalate, silibinin, sodium copper chlorophyllin, and a pharmaceutically acceptable salt or solvate thereof unexpectedly provides benefits for treating at least one condition resulting from envenomation across a broader spectrum of envenomation sources than could be addressed by administration of only the first compound, only the second compound, or any single, species-specific antibody-based antivenom. In some embodiments, the benefit is observed when the second compound is chosen from N-acetyl-L-cysteine, sodium aurothiomalate, or a pharmaceutically acceptable salt or solvate thereof. In some embodiments, the benefit is observed when the second compound is N-acetyl-L-cysteine or a pharmaceutically acceptable salt or solvate thereof. In some embodiments, the benefit is observed when the second compound is sodium aurothiomalate or a pharmaceutically acceptable salt or solvate thereof.

Without being bound by any theory, the combination therapies disclosed herein may target spreading factors widely shared by various venoms, including hyaluronidases, metalloproteinases, and/or phospholipase A2 (PLA2), allowing for treatment efficacy for envenomation from a broad spectrum of species. This efficacy is surprising since the individual compounds are known in the art but not previously indicated for use in combination as an envenomation therapy. Furthermore, the efficacy of the disclosed combinations is also surprising because, in some instances, therapy using only one of the drugs alone may result in inferior treatment of at least one condition associated with envenomation. In some instances, administration of a single drug from the disclosed combination therapy may even result in enhanced spreading factor activity, further demonstrating the surprising inhibition properties of the combination. For example, N-acetyl-L-cysteine alone may enhance PLA2 activities in V. russelli venom by 3-4 fold. Similarly, doxycycline alone may enhance hyaluronidase activity in N. kaouthia and V. russelli venoms.

In some embodiments, the disclosed combination therapies are superior to other mono- or combination therapies such as those using manoalide, which has been reportedly used to inhibit PLA2 toxin from Pachyornis australis. Fatehi et al., Toxicon, 1995. 33(12): p. 1633-43.

In some embodiments, the combination provides added treatment benefits when used in combination with antibody antivenom therapy over what could be obtained by antibody antivenom therapy alone.

Unexpectedly improved conditions after treating envenomation with a combination therapy described herein may include, in some embodiments, one or more of a reduction in edema, blistering, hemorrhage, tissue necrosis, damage to nerve terminals, myotoxicity, cardiotoxicity, and/or alterations in one or more hematological systems. In some embodiments, these improvements are identified by measuring a reduction in one or more myonecrosis and/or serum myonecrosis markers, and/or an alteration in one or more hemorrhagic indicators. In some embodiments, the unexpected improvement may further include a reduction in the risk of morbidity and/or mortality.

Administering a combination therapy described herein may slow venom spreading factors, thus improving survival, and/or it may inhibit or reduce local tissue damage (e.g., via necrotic processes), thus reducing the extent of local tissue damage following envenomation.

### Definitions

The following definitions are used in the present specification. The initial definition provided for a group or term herein applies to that group or term throughout the present specification individually or as part of another group, unless otherwise indicated or implied based on context.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%. All ranges include the endpoints, and the use of the term “or” means “and/or” unless otherwise indicated by context.

The terms “administer”, “administering”, or “administration” are used herein in their broadest sense. These terms refer to any method of introducing to a subject a compound or pharmaceutical composition described herein and can include introducing the compound systemically, locally, or in situ to the subject. For instance, the term includes topical, parenteral, subcutaneous, intraperitoneal, intramuscular, intraarterial, intradermal, and/or intravenous injection of a single compound or a mixture of compounds, as well as pharmaceutically acceptable salts, adjuvants, and the like.

The term “subject” generally refers to an organism to which the compounds or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to humans, mammals and non-mammals, such as non-human primates, mice, rabbits, sheep, dogs, cats, horses, cows, chickens, amphibians, and reptiles, which is to be the recipient of a compound or pharmaceutical composition described herein. Under some circumstances, the terms “subject” and “patient” are used interchangeably herein in reference to a human subject.

The term “effective amount” or “therapeutically effective amount” refers to that amount of a compound or pharmaceutical composition thereof that is sufficient to effect the intended result including, but not limited to, complete or partial treatment, prevention, or amelioration of a condition, as illustrated below. In some embodiments, these terms refer to the amount necessary to disrupt, delay, or prevent the spreading of snake venom and/or spreading factors in the venom. In some embodiments, the “therapeutically effective amount” is the amount that is effective for, e.g., reducing edema, necrosis, inflammation, nerve damage, vascular damage, myotoxicity, cardiotoxicity, and/or blistering. In some embodiments, the “therapeutically effective amount” is the amount that is effective for, e.g., reducing myonecrosis and/or serum myonecrosis markers, and/or altering hemorrhagic indicators or other indicia of envenomation. In one embodiment, the “therapeutically effective amount” is the amount effective to reduce myonecrosis markers, serum myonecrosis markers, and/or hemorrhagic indicators or other indicia of envenomation by at least 25%, or at least 20%, or at least 15%, or at least 10%, and/or the amount effective to reduce hemorrhagic indicators by at least 25%, or at least 20%, or at least 15%, or at least 10%. In some embodiments, a “therapeutically effective amount” is the amount that is effective to reduce the risk of morbidity and/or mortality.

In some embodiments, the “therapeutically effective amount” is the amount that is effective for, e.g., preventing edema, necrosis, nerve damage, vascular damage, myotoxicity, cardiotoxicity, blistering, and/or inflammation from occurring, or from spreading after administration. In some embodiments, the “therapeutically effective amount” is the amount that is effective for, e.g., reducing edema, necrosis, nerve damage, vascular damage, myotoxicity, cardiotoxicity, blistering, and/or inflammation from occurring, or from spreading after administration of a combination therapy, by at least 25%, or at least 20%, or at least 15%, or at least 10%, as compared to no treatment or an antibody-based antivenom treatment.

A therapeutically effective amount can vary depending upon the subject {e.g., the weight and age of the subject), the condition being treated (e.g., the type of venom, the species responsible for the envenomation, the size of the particular animal causing the envenomation, and/or the amount of venom in the subject), the severity of the condition, the manner of administration, and the like, which can readily be determined by one of ordinary skill in the art. A specific dose administered to the subject may vary depending on, for example, the particular pharmaceutical composition, subject and their age and existing health conditions or risk for health conditions, the dosing regimen to be followed, the severity of the condition, whether it is administered in combination with other agents, timing of administration, the tissue to which it is administered, and the physical delivery system in which it is carried. In some embodiments, the total dose may range from about 0.1 ml_ to about 5.0 ml_. In some embodiments, the total dose may range from about 0.5 ml_ to about 2.0 ml_. In certain embodiments, the total dose may be about 0.5 ml_ about 0.75 ml_, about 1 ml_, about 1.25 ml_, about 1.5 ml_, about 1.75 ml_, and about 2 ml_. In some embodiments, the therapeutically effective amount can be injected in one or multiple injections at the same or different injection sites, e.g., using a microderm needle or other suitable delivery device.

As used herein, the terms “treatment”, “treating”, and “ameliorating” are used interchangeably. These terms refer to an approach for obtaining beneficial or desired results including, but not limited to, therapeutic benefit and/or prophylactic benefit. A therapeutic benefit may include partial reduction or complete eradication, or partial or complete amelioration, of the underlying condition being treated (e.g., including neutralization of one or more venom components). Also, a therapeutic benefit may be achieved by the eradication or amelioration (partial or complete) of one or more of the physiological symptoms associated with the underlying condition such that an improvement is observed in the patient, notwithstanding that the patient may still be afflicted by the underlying condition.

In some embodiments, a therapeutically effective amount of a first compound having formula (I), or a pharmaceutically acceptable salt or solvate thereof, and a therapeutically effective amount of a second compound chosen from N-acetyl-L-cysteine and sodium aurothiomalate, or a pharmaceutically acceptable salt thereof, is administered to a subject after envenomation of the subject. In some embodiments, the first compound is a compound having formula (I), or a pharmaceutically acceptable salt or solvate thereof, and the second compound is N-acetyl-L-cysteine, or a pharmaceutically acceptable salt or solvate thereof. In some embodiments, the first compound is a compound having formula (I) and the second compound is sodium aurothiomalate, or a pharmaceutically acceptable salt or solvate thereof.

As used herein, the term “at least one condition associated with envenomation” means a condition, syndrome, symptom, or ailment that results from envenomation by an animal or plant, such as, e.g., envenomation from a snake; bee; stonefish; scorpion; spider; lizard; wasp; hornet; sea urchin; cnidarian such as, e.g., anemone, coral, common or tropical jellyfish; or a toxic plant, such as, e.g., poison ivy, poison sumac, or poison oak. In some embodiments, the at least one condition associated with envenomation is at least one condition associated with a snake bite. In some embodiments, the at least one condition associated with envenomation may be edema, blistering, hemorrhage, tissue necrosis, damage to nerve terminals, myotoxicity, cardiotoxicity, and/or alterations in hematological systems. In some embodiments, the at least one condition associated with envenomation may be identified by an increase in one or more myonecrosis and/or serum myonecrosis markers, and/or an alteration in one or more hemorrhagic indicators. In some embodiments, the at least one condition associated with envenomation may comprise general morbidity and/or mortality.

As used herein, the terms “envenomation” and “envenomed”, which may be used interchangeably, mean any contact with an animal or plant that is the source of the venom in a subject. In some embodiments, the animal may be selected from, e.g., a snake; bee; stonefish; scorpion; spider; lizard; wasp; hornet; sea urchin; and cnidarian such as, e.g., anemone, coral, common and tropical jellyfish. In some embodiments, the plant may be selected from, e.g., a toxic plant, such as, e.g., poison ivy, poison sumac, and poison oak. For example, if a snake attempts to bite a subject and the skin is not broken, however venom makes contact with the subject's skin, then the subject has still suffered from envenomation.

The term, “N-acetyl-L-cysteine” as used herein refers to the compound depicted by the structure:

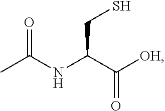

and also identified as CAS #616-91-1.

The term, “sodium aurothiomalate” as used herein, is also known as gold sodium thiomalate and is identified as CAS #12244-57-4.

The terms “salt(s)” and “pharmaceutically acceptable salt(s)”, as used herein, includes acidic and/or basic salts formed with inorganic and/or organic acids and bases. As used herein, the term “pharmaceutically acceptable salt” refers to those salts which are, within the scope of sound medical judgment, suitable for use in contact with the tissues of subjects without undue toxicity, irritation, allergic response and/or the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

Pharmaceutically acceptable salts may be formed with inorganic or organic acids. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Salts may be prepared in situ during the isolation and purification of the disclosed compound, or separately, such as by reacting the compound with a suitable base or acid, respectively. Non-limiting examples of pharmaceutically acceptable salts derived from bases include alkali metal, alkaline earth metal, ammonium and N⁺(Ci-4alkyl)₄ salts. Non-limiting examples of suitable alkali or alkaline earth metal salts include sodium, lithium, potassium, calcium, magnesium, iron, zinc, copper, manganese, and aluminum salts. Further non-limiting examples of suitable pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Non-limiting examples of suitable organic bases from which salts may be derived include primary amines, secondary amines, tertiary amines, substituted amines including naturally occurring substituted amines, cyclic amines, and basic ion exchange resins, such as isopropylamine, trimethylamine, diethylamine, triethylamine, tripropylamine, and ethanolamine. In some embodiments, pharmaceutically acceptable base addition salts can be chosen from ammonium, potassium, sodium, calcium, and magnesium salts.

Solvates of the compounds of the present disclosure are also contemplated herein. The term “solvate” represents an aggregate that comprises one or more molecules of a compound of the present disclosure with one or more molecules of a solvent or solvents. Solvates of the compounds of the present disclosure include, for example, hydrates.

The term “vehicle” as used herein means a pharmaceutically acceptable material, composition or carrier, such as, for example, a liquid or solid filler, diluent, excipient, solvent, or encapsulating material involved in or capable of carrying or transporting the combination of compounds of the present disclosure from one organ, or portion of the body, to another organ, or portion of the body. Each vehicle must be “acceptable” in the sense of being compatible with the other ingredients of the formulation and not injurious to the patient. Non-limiting examples of pharmaceutically acceptable vehicles, vehicles, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

Compositions suitable for parenteral, (e.g., intradermal) administration may comprise at least one more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

### Equivalents

It will be readily apparent to those skilled in the art that other suitable modifications and adaptations of the methods described herein may be made using suitable equivalents without departing from the scope of the disclosure herein.

Having now described the disclosed methods and compositions in detail, the same will be more clearly understood by reference to the following examples, which are included for purposes of illustration only and are not intended to be limiting.

### EXAMPLES

### Example 1

### Assessment of Inhibition Activity Using a Single Spreading Factor Inhibitor

### Materials

EnzChek® Phospholipase A 2 and EnzChek® Gelatinase/Collagenase assay kits were purchased from ThermoFisher. Cromolyn disodium salt, doxycycline hyclate, and hyaluronic acid were purchased from Santa Cruz Biotechnolgy. Other chemicals included sodium aurothiomalate (Alfa Aesar); sodium acetate, trihydrate (J.T. Baker); bovine albumin (Amresco); sodium phosphate, monobasic, monohydrate (Calbiochem); manoalide (ChemCruz), and bovine testes hyaluronidase (MP Biomedicals, LLC). All other chemicals, venoms (Sigma Aldrich #V2501 for Vipera russelli venom and Sigma Aldrich #V9125 for Naja naja kaouthia venom), and enzymes were purchased from Sigma Aldrich. All fluoromoetric and turbidity measurements determined using a Biotek Synergy HT spectrophotometer.

### Venom Reconstitution

Lyophilized N. kaouthia and V. russelli {Daboia russelli) venoms were reconstituted in ice cold 0.9% sodium chloride (0.22 μm filtered) to 10 mg/mL and 8 mg/mL, respectively. Samples were briefly centrifuged to remove residual particular matter, aliquoted, and stored at −20° C.

### Hyaluronidase Activity Assay

Hyaluronidase activity was assessed using the Enzymatic Assay of Hyaluronidase (3.2.1.35) protocol from Sigma Aldrich. Briefly, equal volumes of venom were incubated with 0.03% hyaluronic acid solution (0.03% (w/v) hyaluronic acid in 300 mM sodium phosphate (approximately pH 5.35)) for 1.5 hours at 37° C. A reaction aliquot was then added to a 5-fold volume of acidic albumin solution (24 mM sodium acetate, 70 mM acetic acid, 0.1% BSA adjusted to approximately pH 3.75), allowed to stand for 10 minutes, and then absorbance at 600 nm was measured. Data was converted to percent transmission (% T) and adjusted for background signals. Venom hyaluronidase activities were determined by creating a standard curve with bovine testes hyaluronidase.

For inhibition experiments, 6.75 μg N. kaouthia or 15 μg V. russelli venom was pre-incubated with the specified spreading factor inhibitor(s) for 5-10 minutes before adding an equal volume of the reaction to the hyaluronic acid solution. Data was converted to % T, adjusted for background signals, and then normalized to the venom-only controls.

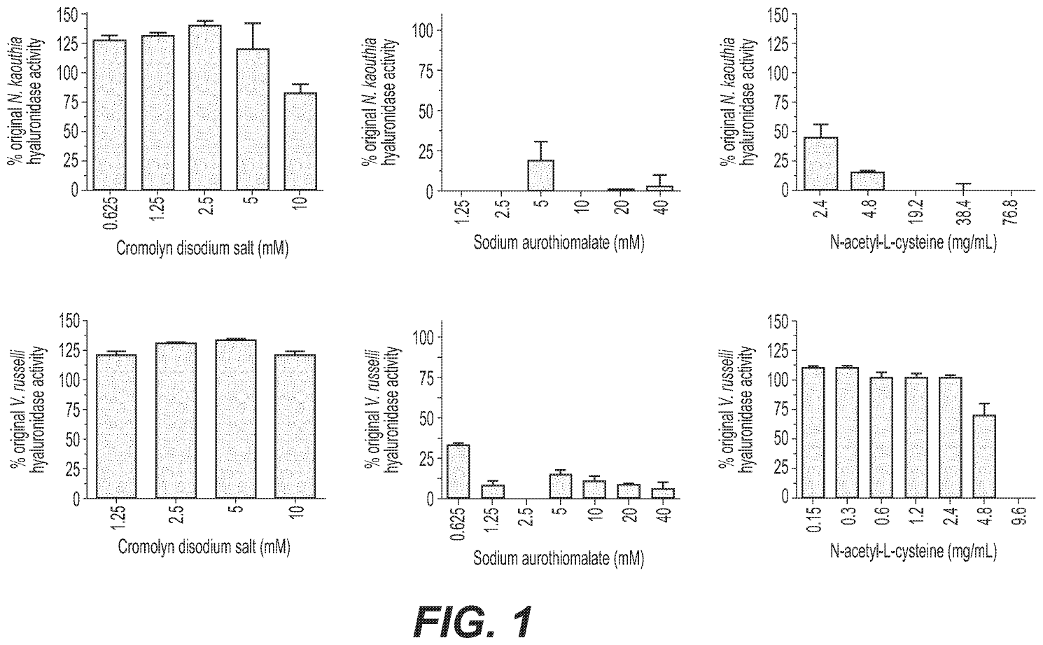

FIG. 1 shows the inhibition, by compositions of the present disclosure, of N. kaouthia and V. russelli crude venom hyaluronidase activities.

### Gelatinase Activity Assay

EnzChek® Gelatinase/Collagenase assay kits were used according to the manufacturer's recommendations. Briefly, equal volumes of venom solutions were incubated with 1.0 mg/mL DQ gelatin and fluorometric analysis was performed for at least three hours, using excitation and emission wavelengths of 485(20) nm and 528(20) nm, respectively. Venom activity was determined by creating a standard curve with Clostridium histolyticum collagenase activity.

For inhibition experiments, 30 μg N. kaouthia or V. russelli venom was pre-incubated with the specified spreading factor inhibitor(s) for 5-10 minutes before adding an equal volume of the reaction to the 1.0 mg/mL DQ gelatin solution. Fluorometric readings were measured over the course of five hours, with the five hour time points being used to determine the percent inhibition. Data was adjusted for background signals and then normalized to the venom-only controls.

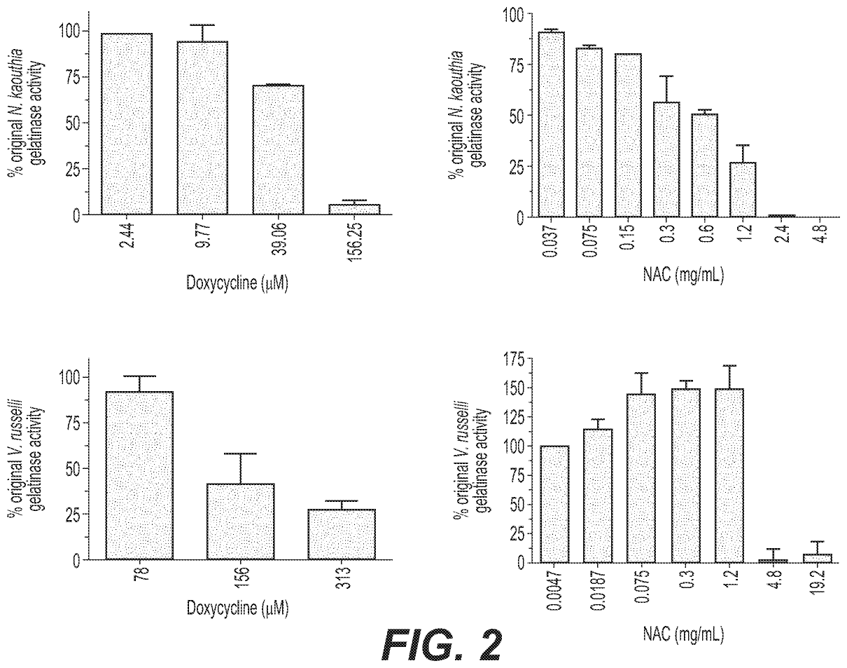

FIG. 2 shows the inhibition of N. kaouthia and V. russelli crude venom gelatinase activities.

### Phospholipase A₂ Activity Assay

EnzChek® Phospholipase A 2 assay kits were used according to the manufacturer's recommendations. Briefly, equal volumes of venom solutions were incubated with PLA2 substrate and fluorometric analysis was performed over the course of 20 minutes. Venom activity was determined by creating a standard curve based upon PLA2 from honey bee venom.

For inhibition experiments, 0.4 μg N. kaouthia or 0.2 μg V. russelli venom was pre-incubated with the specified spreading factor inhibitor(s) for 5-10 minutes before adding the reaction to an equal volume of PLA2 substrate. Fluorometric analysis was performed over 20 minutes, with the 10 minute time point values being used to determine the percent inhibition. Background values were subtracted from all samples, and data was normalized to the venom-only controls. Excitation and emission wavelengths for these experiments were 485(20) nm and 528(20) nm, respectively.

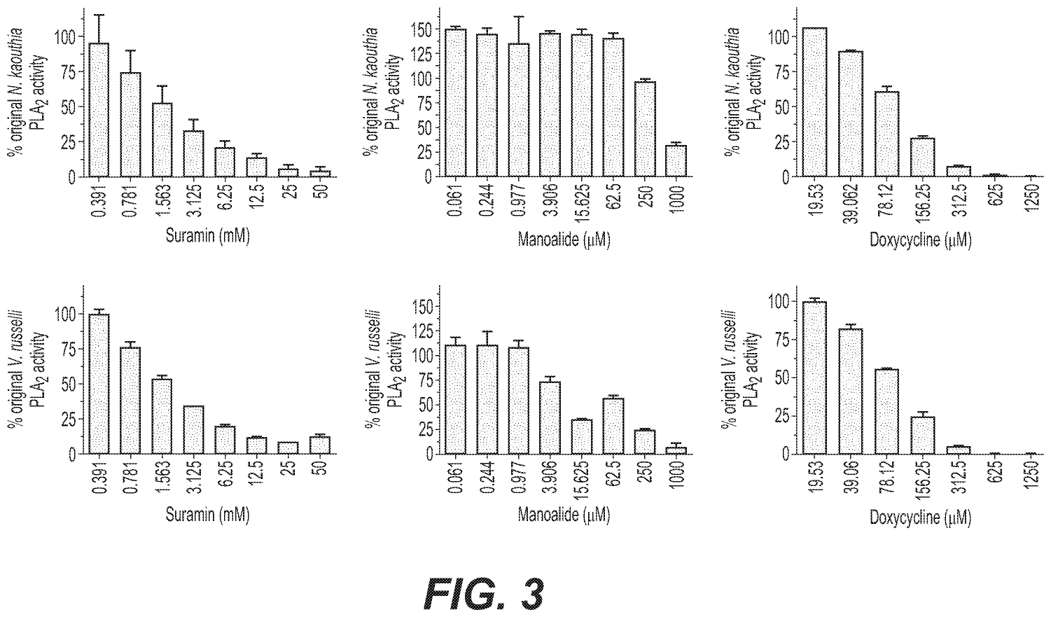

FIG. 3 shows the inhibition of N. kaouthia and V. russelli crude venom PLA2 activities.

Data are shown as the mean and standard deviation (SD) of at least duplicate readings. The half maximal inhibitory concentration (IC50) values were determined using GraphPad Prism. Table 1 summarizes the inhibitory activity of various spreading factor inhibitors for N. kaouthia and V. russelli crude venom extracts.

TABLE 1
Summary of inhibitory values. Values marked with an asterisk (*)
represent data obtained according to Example 2. All other values represent
data obtained according to Experiment 1.

| Inhibitor | SFI activity | Value | N. kaouthia venom | V. russelli venom |
|---|---|---|---|---|
| Sodium | Hyaluronidase | IC₅₀ | 41.98 μM | 146.89 μM |
| aurothiomalate | | ≥90% | 136.30 μM | 373.25 μM |
| | | inhibition | | |
| | Gelatinase* | IC₅₀ | 47.04 ± 30.39 μM* | N/A |
| | | ≥90% | >208 μM* | N/A |
| | | inhibition | | |
| Cromolyn | Hyaluronidase | IC₅₀ | Not determined | Not determined |
| disodium salt | | ≥90% | Not determined | Not determined |
| | | inhibition | | |
| Doxycycline | Gelatinase | IC₅₀ | 101.62 ± 22.81 μM* | 127.51 ± 11.49 μM |
| | | ≥90% | 298.62 ± 34.55 μM* | >313 μM |
| | | inhibition | | |
| | PLA₂ | IC₅₀ | 242.52 ± 168.02 μM | 167.40 ± 59.39 μM |
| | | ≥90% | 237.57 ± 23.78 μM | 225.23 ± 23.42 μM |
| | | inhibition | | |
| Manoalide | PLA₂ | IC₅₀ | 776.8 μM | 31.41 μM |
| | | ≥90% | >1000 μM | 1000 μM |
| | | inhibition | | |
| N-acetyl-L- | Hyaluronidase | IC₅₀ | 4.503 mg/mL | ~4.989 mg/mL |
| cysteine | | ≥90% | 4.8 mg/mL | 6.0-9.6 mg/mL |
| | | inhibition | | |
| | Gelatinase | IC₅₀ | 1.85 mg/mL | 1.87 mg/mL |
| | | ≥90% | 1.32 mg/mL | 2.31 mg/mL |
| | | inhibition | | |
| Suramin | PLA₂ | IC₅₀ | 1.515 ± 0.528 mM | 2.010 mM |
| | | ≥90% | 12.5 mM | 12.5 mM |
| | | inhibition | | |

### Example 2

### Activity of Combinations of Spreading Factor Inhibitors

Three drug combinations were selected to test based on their capacity to inhibit hyaluronidase, gelatinase (metalloproteinase), and/or PLA2 activities found in both N. kaouthia and V. russeiii venoms, as described in Example 1: sodium aurothiomalate and doxycycline; doxycycline and N-acetyl-L-cysteine; and sodium aurothiomalate and manoalide.

### Sodium Aurothiomalate and Doxycycline

Snake venom samples were incubated with increasing concentrations of sodium aurothiomalate and/or doxycycline for 5-10 minutes prior to assaying the venom hyaluronidase, gelatinase, and PLA2 activities. As observed in Example 1, sodium aurothiomalate inhibited N. kaouthia and V. russeiii venom hyaluronidase activities, whereas doxycycline inhibited the venom gelatinase and PLA2 activities. Interestingly, venoms incubated with only sodium aurothiomalate alone also displayed a moderate, but statistically significant increase in PLA2 activities. Similarly, pre-incubation with only doxycycline resulted in a significant increase in N. kaouthia venom hyaluronidase activity. In some experimental replicates, pre-incubation with doxycycline also caused an increase in V. russeiii hyaluronidase activity.

Pre-incubation, similar to Example 1, with 208 μm sodium aurothiomalate and 313 μM doxycycline was sufficient to reduce hyaluronidase, gelatinase, and PLA2 activities to ≤10% of the uninhibited venom, with the exception of V. russeiii hyaluronidase activities, which were reduced to 50% of the original crude venom activity. Increasing the amount of sodium aurothiomalate from 208 μM to 625 μm and/or increasing the amount of doxycycline from 313 μM to 626 μM did not statistically improve the ability of the combination to inhibit N. kaouthia or V. russeiii spreading factor activities (data not shown).

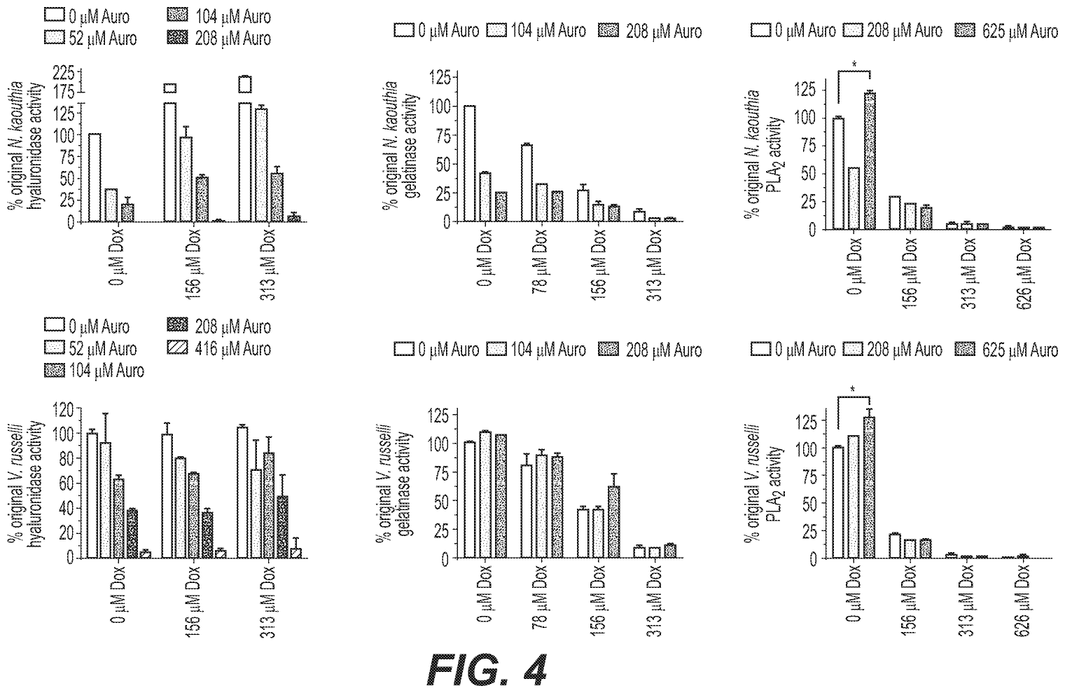

FIG. 4 shows that the combination of sodium aurothiomalate and doxycycline significantly reduced all three spreading factor activities, including those that were elevated in the presence of the individual drugs.

### Doxycycline and N-acetyl-L-cysteine

Similar to the results obtained in Experiment 1, doxycycline inhibited both N. kaouthia and V. russeiii gelatinase and PLA2 activities. N-acetyl-L-cysteine alone inhibited the venom hyaluronidase and gelatinase activities, and complete hyaluronidase inhibition of crude V. russeiii venom required significantly more N-acetyl-L-cysteine than necessary to completely inhibit N. kaouthia crude venom hyaluronidase activity. Conversely, pre-incubation with N-acetyl-L-cysteine alone consistently enhanced PLA2 activities in both N. kaouthia and V. russeiii venoms.

Pre-incubation, similar to Example 1, with 156 μM doxycycline and 6 mg/ml_ N-acetyl-L-cysteine was sufficient to reduce hyaluronidase, gelatinase, and PLA2 activities to <10% of the uninhibited venom, with the exception of V. russeiii hyaluronidase activities, which were reduced to approximately 50% of the original crude venom activity. Notably, doxycycline had no impact on the ability of N-acetyl-L-cysteine to inhibit venom hyaluronidase activities. The combination treatment also appeared to additively inhibit venom gelatinase activities. Without being bound by theory, this may be due to the fact that both N-acetyl-L-cysteine and doxycycline display anti-gelatinase activities. Although 1.5-6.0 mg/mL N-acetyl-L-cysteine enhanced N. kaouthia and V. russeiii venom PLA2 activities, this effect could be overcome with administration of 156 μM doxycycline.

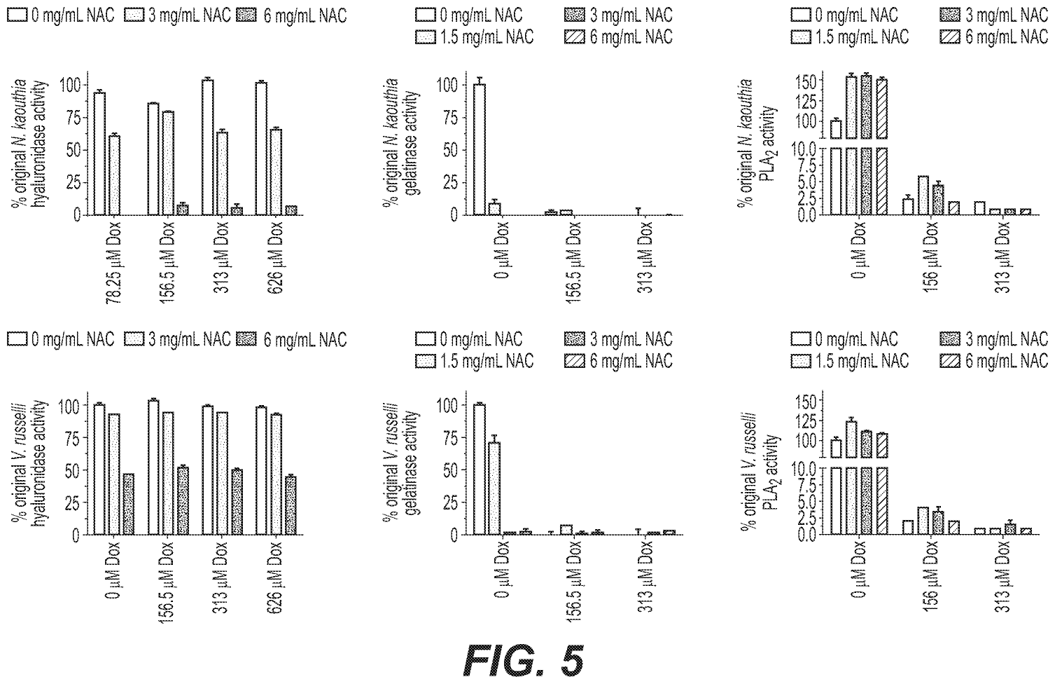

FIG. 5 shows the combined inhibitory activity of doxycycline and N-acetyl-L-cysteine toward N. kaouthia and V. russeiii spreading factors.

### Sodium Aurothiomalate and Manoalide

The combined effect of sodium aurothiomalate and manoalide on V. russeiii venom hyaluronidase and PLA2 activities was determined, similar to the procedure used in Example 1. When sodium aurothiomalate concentrations equal to or greater than 2.5 mM were combined with 12 μM manoalide, N. kaouthia PLA2 activities increased. Notably, 12 μM manoalide was significantly less than what was necessary to inhibit N. kaouthia venom PLA2 activity.

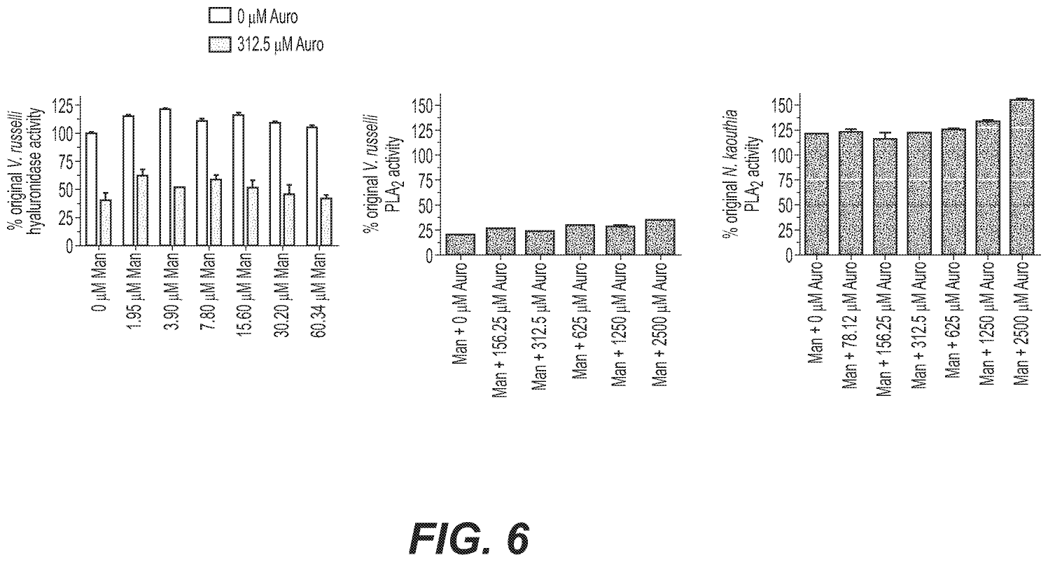

FIG. 6 shows the inhibitory activity of the combination of sodium aurothiomalate and manoalide toward V. russeiii hyaluronidase activities and V. russeiii and N. kaouthia PLA2 activities.

In summary, it was found that the administration of the compounds alone may have undesirable effects. For example, administration of doxycycline alone may result in increased hyaluronidase activity in N. kaouthia and V. russeiii venom; administration of sodium aurothiomalate alone may result in increased PLA2 activity in N. kaouthia and V. russeiii venom; and administration of N-acetyl-L-cysteine alone may result in increased PLA2 activity in V. russeiii and N. kaouthia venoms.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described accordingly, all suitable modifications and equivalents may be resorted to, falling within the scope of the present disclosure.

1. A method of treating at least one condition associated with envenomation in a subject comprising administering to a subject in need thereof a therapeutically effective amount of a first compound having formula (I): 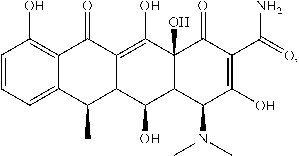 [SMILES: C[C@H]1c2cccc(O)c2C(=O)C2=C(O)[C@]3(O)C(=O)C(C(N)=O)=C(O)[C@@H](N(C)C)C3[C@@H](O)C21]
or a pharmaceutically acceptable salt or solvate thereof;
a therapeutically effective amount of a second compound chosen from N-acetyl-L-cysteine, sodium aurothiomalate, silibinin, sodium copper chlorophyllin, and pharmaceutically acceptable salts and solvates thereof; and
at least one pharmaceutically acceptable vehicle.

2. The method according to claim 1, wherein the condition associated with envenomation is edema, blistering, hemorrhage, tissue necrosis, damage to nerve terminals, myotoxicity, cardiotoxicity, alteration in a hematological system, increase in a myonecrosis marker, increase in a serum myonecrosis marker, and/or increase in a hemorrhagic indicator.

3. The method according to claim 1, wherein the first compound and the second compound are administered within 96 hours, 48 hours, 24 hours, 12 hours, 6 hours, one hour, or less of envenomation.

4. The method according to claim 1, wherein the first compound and the second compound are administered within 50 minutes, 40 minutes, 30 minutes, 20 minutes, 10 minutes, 5 minutes, or less of envenomation.

5. The method according to claim 1, wherein the first compound and the second compound are administered within 30 minutes of envenomation.

6. The method according to claim 1, wherein the first compound and the second compound are administered within 10 minutes of envenomation.

7. The method according to claim 1, wherein the first compound and the second compound are administered within 5 minutes of envenomation.

8. (canceled)

9. The method according to claim 1, wherein the first compound is administered at the same time as the second compound.

10. The method according to claim 1, wherein the first compound is administered before the second compound.

11. The method according to claim 1, wherein the first compound is administered after the second compound.

12. The method according to claim 1, further comprising administering an additional therapeutic agent to the subject in need thereof before, at the same time as, or after the administering of the first compound and the second compound, optionally wherein the additional therapeutic agent comprises an antibody-based antivenom.

13. The method according to claim 12, wherein the antibody-based anti-venom is administered 96 hours, 24 hours, 12 hours, 6 hours, one hour, or less before and/or after administering the first compound and the second compound.

14. The method according to claim 13, wherein the antibody-based anti-venom is administered within 50 minutes, 40 minutes, 30 minutes, 20 minutes, 10 minutes, 5 minutes, or less before and/or after administering the first compound and the second compound.

15. The method according to claim 1, wherein the therapeutically effective amount of the first compound ranges from about 0.5 g to about 1.5 g and the therapeutically effective amount of the second compound ranges from about 18 g to about 54 g or ranges from about 0.25 g to about 0.75 g.

16. The method according to claim 1, wherein the envenomation is from a snake, a bee, a stonefish, a scorpion, a spider, a lizard, a wasp, a hornet, a sea urchin, a cnidarian, a toxic plant, and/or a pathogenic bacteria.

17. The method according to claim 1, wherein the envenomation is from a bite of a snake, and optionally wherein the snake is of the Elapidae, Viperidae, Colubridae, Hydrophiidae, or Atractaspididae family.

18. The method according to claim 1, wherein the envenomation is from a bite of a snake, and optionally wherein the snake is of the Elapidae or Viperidae family.

19. The method according to claim 1, wherein the first compound is: 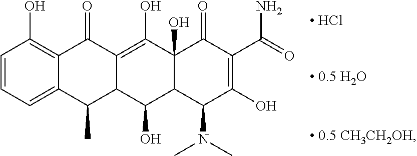 [SMILES: C[C@H]1c2cccc(O)c2C(=O)C2=C(O)[C@]3(O)C(=O)C(C(N)=O)=C(O)[C@@H](N(C)C)C3[C@@H](O)C21 ; "· HCl"]
or a pharmaceutically acceptable salt or solvate thereof.

20. The method according to claim 1, wherein the second compound is N-acetyl-L-cysteine or a pharmaceutically acceptable salt or solvate thereof.

21. The method according to claim 1, wherein the second compound is sodium aurothiomalate or a pharmaceutically acceptable salt or solvate thereof.

22. The method of claim 1, comprising administering therapeutically effective amounts of the first and second compounds to reduce one or more conditions associated with envenomation as compared to no treatment or treatment with an antibody-based antivenom alone, and optionally wherein the one or more conditions associated with envenomation comprises edema, blistering, hemorrhage, tissue necrosis, damage to nerve terminals, myotoxicity, cardiotoxicity, alteration in a hematological system, increase in a myonecrosis marker, increase in a serum myonecrosis marker, and/or increase in a hemorrhagic indicator.

23. The method of claim 22, wherein administering therapeutically effective amounts of the first and second compounds inhibits or slows the spread of venom and/or inhibits or slows local tissue damage at a site of envenomation, as compared to no treatment or treatment with an antibody-based antivenom alone.

24. A pharmaceutical composition comprising:
a first compound having formula (I): 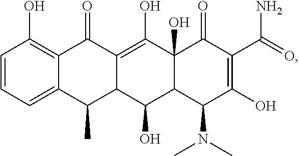 [SMILES: C[C@H]1c2cccc(O)c2C(=O)C2=C(O)[C@]3(O)C(=O)C(C(N)=O)=C(O)[C@@H](N(C)C)C3[C@@H](O)C21]
or a pharmaceutically acceptable salt or solvate thereof;
a second compound chosen from N-acetyl-L-cysteine and sodium aurothiomalate or a pharmaceutically acceptable salt or solvate thereof; and
at least one pharmaceutically acceptable vehicle.

25. The pharmaceutical composition according to claim 24, wherein the first compound is: 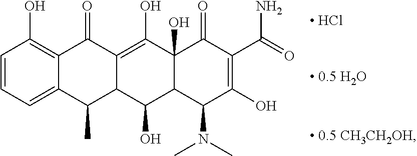 [SMILES: C[C@H]1c2cccc(O)c2C(=O)C2=C(O)[C@]3(O)C(=O)C(C(N)=O)=C(O)[C@@H](N(C)C)C3[C@@H](O)C21 ; "· HCl"]
or a pharmaceutically acceptable salt or solvate thereof.

26. The pharmaceutical composition according to claim 24, wherein the second compound is N-acetyl-L-cysteine or a pharmaceutically acceptable salt or solvate thereof.

27. The pharmaceutical composition according to claim 24, wherein the second compound is sodium aurothiomalate or a pharmaceutically acceptable salt or solvate thereof.

28. The pharmaceutical composition according to claim 24, wherein the first compound and the second compound are each present in a therapeutically effective amount.

29. The pharmaceutical composition according to claim 28, wherein the therapeutically effective amount of the first compound ranges from about 0.5 g to about 1.5 g and the therapeutically effective amount of the second compound ranges from about 18 g to about 54 g or ranges from about 0.25 g to about 0.75 g.

30. A kit comprising: a first compound having formula (I): 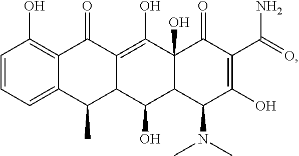 [SMILES: C[C@H]1c2cccc(O)c2C(=O)C2=C(O)[C@]3(O)C(=O)C(C(N)=O)=C(O)[C@@H](N(C)C)C3[C@@H](O)C21]
or a pharmaceutically acceptable salt or solvate thereof; and
a second compound chosen from N-acetyl-L-cysteine and sodium aurothiomalate or a pharmaceutically acceptable salt or solvate thereof.

31. The kit according to claim 30, wherein the first compound is: 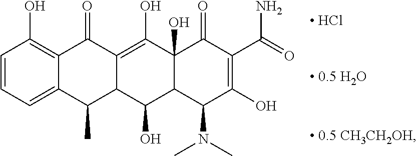 [SMILES: C[C@H]1c2cccc(O)c2C(=O)C2=C(O)[C@]3(O)C(=O)C(C(N)=O)=C(O)[C@@H](N(C)C)C3[C@@H](O)C21 ; "· HCl"]
or a pharmaceutically acceptable salt or solvate thereof.

32. The kit according to claim 30, wherein the second compound is N-acetyl-L-cysteine and pharmaceutically acceptable salts and solvates thereof.

33. The kit according to claim 30, wherein the second compound is sodium aurothiomalate and pharmaceutically acceptable salts and solvates thereof.

34.-43. (canceled)
